(19) United States
(12) Patent Application Publication
(10) Pub. No.: US 2018/0250172 A1
(43) Pub. Date: Sep. 6, 2018

(54) ROUND TIP VAGINAL INSERT SYSTEM AND METHOD FOR ABSORBING SEXUAL SECRETIONS

(71) Applicant: Darvyaire Turner, Chicago, IL (US)

(72) Inventor: Darvyaire Turner, Chicago, IL (US)

(21) Appl. No.: 15/913,624

(22) Filed: Mar. 6, 2018

Related U.S. Application Data
(60) Provisional application No. 62/467,325, filed on Mar. 6, 2017.

(51) Int. Cl. A61F 13/20; A61F 6/06; A61F 13/26; A61F 13/34; A61M 31/00

(52) U.S. Cl. CPC A61F 13/2002; A61F 6/06; A61F 13/2074; A61F 2013/15097; A61F 13/34; A61M 31/007; A61F 13/266

(57) ABSTRACT

A Round Tip Vaginal Insert System and Method For Absorbing Sexual Secretions is described herein. The present disclosure may be implemented as a system, comprising: an outer receptacle, wherein the outer receptacle further comprises a bulb shaped head and a cylindrical shaft; an absorbent element, wherein the absorbent element comprises a cord; and a loading cylinder. The present disclosure may be implement as a method, comprising inserting an absorbent element into a outer receptacle via a loading cylinder; inserting the outer receptacle into a body cavity; compressing the loading cylinder to insert the absorbent element into the body cavity; and removing the loading cylinder and outer receptacle from the body cavity. The present disclosure may be an apparatus comprising an outer receptacle, a loading cylinder, and a head of the loading cylinder fitting into the outer receptacle in an interlocking connection that allows the loading cylinder to slide vertically up and down the outer receptacle.

### RELATED APPLICATION(S)

Under provisions of 35 U.S.C. § 119(e), the Applicant claims the benefit of U.S. provisional application No. 62/467,325, filed Mar. 6, 2017, which is incorporated herein by reference.

It is intended that each of the referenced applications may be applicable to the concepts and embodiments disclosed herein, even if such concepts and embodiments are disclosed in the referenced applications with different limitations and configurations and described using different examples and terminology.

### TECHNICAL FIELD

This disclosure relates generally to feminine hygiene products.

### BACKGROUND

Feminine hygiene products inserted into the female body cavity have served the purpose to collect fluids discharged during menstruation. While a variety of solutions exist for the collection of discharged blood, secretions, and tissue debris from the uterus that recurs in non-pregnant women, there is no effective solution for collecting leaking residue and secretions remaining in the vaginal cavity post sexual intercourse. While panty liners are used to capture post coital secretions, this solution fails to capture post coital fluids leaking from the vaginal cavity resulting in unpleasant feelings, odors, unwanted fluids, and messy surroundings after sexual intercourse. Fluids include but are not limited to secretions naturally and artificially emanating from the female and/or male participants.

The above-described background relating to feminine hygiene products is merely intended to provide a contextual overview of this type of technology, and is not intended to be exhaustive. Other context regarding such products may become further apparent upon review of the following detailed description.

### BRIEF DESCRIPTION OF THE DRAWINGS

Non-limiting and non-exhaustive embodiments of the subject disclosure are described with reference to the following figures, wherein like reference numerals refer to like parts throughout the various views unless otherwise specified.

FIG. 1A illustrates an example schematic of an apparatus comprising an embodiment of an outer receptacle consistent with this disclosure.

FIG. 1B illustrates an example schematic of an apparatus comprising a loading cylinder consistent with this disclosure.

FIG. 2 illustrates an example schematic of a system comprising an outer receptacle, a loading element, and an absorbent element consistent with this disclosure.

FIG. 3 illustrates an example schematic of an apparatus comprising an embodiment of an outer receptacle consistent with this disclosure.

FIG. 4 illustrates an example schematic of a method of using the system consistent with this disclosure.

### DETAILED DESCRIPTION

In the following description, numerous specific details are set forth to provide a thorough understanding of various embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment,” or “an embodiment,” means that a particular feature, structure, or characteristic described in connection with the embodiment is included in at least one embodiment. Thus, the appearances of the phrase “in one embodiment,” “in one aspect,” or “in an embodiment,” in various places throughout this specification are not necessarily all referring to the same embodiment. Furthermore, the particular features, structures, or characteristics may be combined in any suitable manner in one or more embodiments.

As an overview of the various embodiments presented herein, to correct for the above identified deficiencies and other drawbacks of feminine hygiene products, various embodiments are described herein to facilitate the collection of fluids including but not limited to post coital fluids, fluids produced during masturbation, and fluids occurring naturally or otherwise.

As stated above fluids, leaking residue, blood, and secretions remaining in the vaginal cavity post coitus present a hygiene problem. The present disclosure addresses this concern and presents additional solutions. In an embodiment of the present disclosure, an apparatus comprising an outer receptacle 101, an absorbent element 205, and a loading cylinder 110 are used to provide absorption and protection after sexual intercourse. The system and apparatus may be used before and/or after sexual intercourse, masturbation, or other sexual activity. In some embodiments, the system and apparatus may be used during sexual intercourse or masturbation. In some embodiments, the system and apparatus may be used to absorb fluids generated in the vaginal cavity resulting from sexual activity and sensual touching away from the vaginal cavity.

A simplified overview is provided herein to help enable a basic or general understanding of various aspects of exemplary, non-limiting embodiments that follow in the more detailed description and the accompanying drawings. This overview is not intended, however, as an extensive or exhaustive overview. Instead, the purpose of this overview is to present some concepts related to some exemplary non-limiting embodiments in simplified form as a prelude to more detailed descriptions of the various embodiments that follow in the disclosure.

Described herein are systems, methods, articles of manufacture, and other embodiments or implementations that may facilitate the use of an absorbent element 205 for absorption of post coital secretions released from the vaginal cavity. The insert may function as a low absorbency tampon. The absorbent element 205 may also function as a smaller compact tampon. The absorbent element 205 may also function as a contraceptive or birth control device. The absorbent element 205 may also function as a device for medical prevention of sexually transmitted disease and infection. The absorbent element 205 may be implemented in connection with any other types of contraceptives, medical devices, and feminine hygiene products, such as an intrauterine device (IUD), a birth control pill, a condom, a sponge, a gel, a foam, a cream, a Depo-Provera™ birth control shot, a spermicide, a diaphragm or the like. The absorbent element 205 may exist as part of a system further comprising an outer receptacle 101 and a loading cylinder 110.

A variety of feminine hygiene products in the market such as tampons suffer from risks of causation of toxic shock syndrome (TSS). In fact higher-absorbency tampons increase the risk of TSS. Additionally, tampons are not suitable for use outside of a woman's menstruation cycle diminishing the product's usefulness in post coital situations. However, the embodiments of the present disclosure presented herein provide several advantages such as a tremendous reduction of the risk of toxic shock syndrome (TSS). Additionally, embodiments of the present disclosure provide several advantages including providing contraceptive protection, a barrier, a spermicide, effective absorption, and STI prevention.

In various embodiments, an outer receptacle 101 may be used in conjunction with a loading cylinder 110, and an absorbent element 205 with a cord 210 attached.

According to one embodiment, described herein is an apparatus for a higher absorbency rate. The apparatus can comprise outer receptacle 101, outer receptacle 300, loading cylinder 110, and absorbent element 205.

According to another embodiment, described herein is a method for facilitating absorbency of bodily fluids post coitus or masturbation. The method can comprise facilitating the absorption of post coital fluids in the vaginal cavity utilizing but not limited to the steps comprising: inserting an absorbent element 205 into a outer receptacle 101 via a loading cylinder 110; inserting the outer receptacle 101 into a body cavity; compressing the loading cylinder 110 to insert the absorbent element 205 into the body cavity; placing the absorbent element 205 in the body cavity through the opening in the bulb shaped head 102 of the outer receptacle 101; removing the loading cylinder 110 from the body cavity; and removing the outer receptacle 101 from the body cavity. Once the fluids are absorbed, removing the absorbent element 205 from the body cavity utilizing the cord 210 which may be a component of the absorbent element 205.

According to yet another embodiment, described herein is an apparatus for facilitating absorbency of post coital bodily fluids in the vaginal cavity. The apparatus can comprise an absorbent element 205 configured to absorb fluids; wherein the absorbent element 205 further comprises at least one of: a spermicide, a contraceptive, and a medical chemical for disease prevention.

In another embodiment, the apparatus may comprise an absorbent element 205 wherein the absorbent element 205 may comprise a sponge, natural cotton, organic cotton, synthetic cotton, cotton, bead, gel, chemical compound, composite element, chemical composition, paste, or the like. The absorbent element 205 may contain a spermicide such as nonoxynol-9, spermicidal jelly, cream, foam, tablets, suppositories, sponge, film, plant based spermicides, natural spermicides, organic spermicides, oils, fats, ghee, and the like.

The absorbent element 205 when inserted into the vaginal cavity prior to sexual intercourse may absorb sperm, bodily fluids, secretions, and the like released during coitus or sexual activity. As a contraceptive, the absorbent element 205 may act as a barrier keeping sperm, semen, ejaculate, and other bodily fluids from reaching the cervix, uterus, or fallopian tubes greatly reducing the risk of pregnancy associated with coitus. In other embodiments, the absorbent element 205 may absorb fluids including but not limited to blood and urine from incontinence.

Panty liners can capture the odors in the vaginal cavity area and cause an uncomfortable residue to accumulate against the vaginal cavity area. Existing tampons are developed for use during a woman's menstrual cycle. With existing tampons, the size, padding, material, and composition make it difficult and uncomfortable for any use during post sexual intercourse occasions. Additionally, existing tampons are not specifically designed to be utilized outside of absorbing menstruation fluids. The present disclosure improves upon these limitations by providing a solution specifically designed for absorbing post coital fluids. The present disclosure utilizes components that provide freshness, comfort, hygienic cleanliness, during coitus and after by absorbing the secretions before they exit the vaginal cavity. Thus, relieving the discomfort of residue and secretions against the vaginal cavity and the odor associated with it.

In a general overview of the present disclosure and a general method of using the system and apparatus of the present disclosure follows including: Insert the Round tip applicator, or outer receptacle 101 into the vaginal cavity after sexual intercourse or one or more post coital sessions. With your fingers hold the insert by the grooved rings and push the inner tube or loading cylinder 110 into the outer tube or outer receptacle 101 to absorbent element 205 or low absorbency tampon, into the vaginal cavity. Remove the outer receptacle 101 and loading cylinder 110 tubes from the vaginal cavity leaving absorbent element 205 inside the vaginal cavity and the removal cord 210, a component of the absorbent element 205, hanging from the vaginal cavity.

An overview of a general relationship between the components is described herein as follows: The absorbent element 205 is held in the outer receptacle 101. The grooved rings around the bottom of the outer receptacle 101 are used to grip the outer receptacle 101 while pushing or loading the absorbent element 205 into the vaginal cavity using the loading cylinder 110. The loading cylinder 110 is used as a loader to push the absorbent element 205 into the vaginal cavity and release it from the outer receptacle 101 through the opening at the end of the bulb shaped head 102 of the outer receptacle 101. The removal cord 210 is a component of the absorbent element 205 and may extend long enough to hang outside of the vaginal cavity.

In another embodiment, the loading cylinder 110 may be attached or connected to the outer receptacle 101. This may be configured to be a more compact embodiment of the outer receptacle 101 containing the loading cylinder 110 within it. The loading cylinder 110 and outer receptacle 101 may be configured to push the absorbent element 205, from the outer receptacle 101 into the vaginal cavity after loading the absorbent element 205 into the outer receptacle 101 from the top.

In yet another embodiment, the absorbent element 205 may be inserted into the vaginal cavity by hand using fingers and leaving the removal cord 210 component of the absorbent element 205 hanging from the vaginal cavity.

A general step by step process of how the present disclosure functions is included as follows: First, insert an absorbent element 205 into an outer receptacle 101 into the vaginal cavity after sexual intercourse. A user may use his or her fingers to hold the outer receptacle 101 by the grooved rings and push the loading cylinder 110 into the outer receptacle 101 to release the absorbent element 205 into the vaginal cavity. Remove the loading cylinder 110 and the outer receptacle 101 from the vaginal cavity, leaving the absorbent element 205 inside the vaginal cavity with the cord 210 component of the absorbent element 205 hanging from the vaginal cavity. The insert, tampon, or absorbent element 205 will absorb the secretions from sexual intercourse that remain in the vaginal cavity.

As stated above, there are various embodiments or combinations to achieve the desired outcome by combining the parts of the system listed in the aforementioned steps. These and other embodiments or implementations are described in more detail below with reference to the drawings.

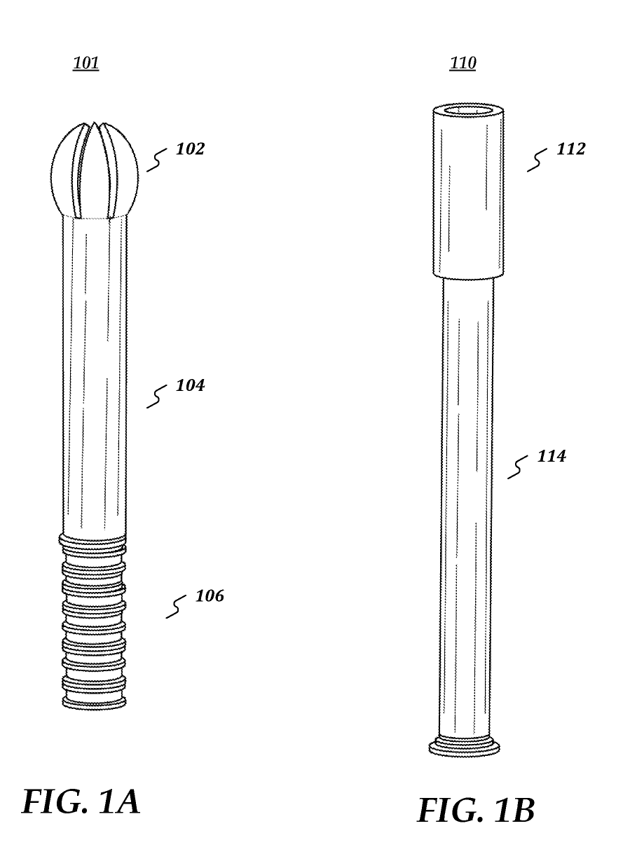

FIGS. 1-4 illustrate apparatuses, systems, and methods that facilitate production of a round tip vaginal insert for sexual secretions consistent with the present disclosure. For simplicity of explanation, the methods are depicted and described as a series of acts. It is to be understood and appreciated that the various embodiments are not limited by the acts illustrated and/or by the order of acts. For example, acts can occur in various orders and/or concurrently, and with other acts not presented or described herein. Furthermore, not all illustrated acts may be required to implement the methods. In addition, the methods could alternatively be represented as a series of interrelated states via a state diagram or events. Additionally, the methods described hereafter are capable of being stored on an article of manufacture.

The above description of illustrated embodiments of the subject disclosure, including what is described in the Abstract, is not intended to be exhaustive or to limit the disclosed embodiments to the precise forms disclosed. While specific embodiments and examples are described herein for illustrative purposes, various modifications are possible that are considered within the scope of such embodiments and examples, as those skilled in the relevant art can recognize.

In this regard, while the subject matter has been described herein in connection with various embodiments and corresponding FIGs, where applicable, it is to be understood that other similar embodiments can be used or modifications and additions can be made to the described embodiments for performing the same, similar, alternative, or substitute function of the disclosed subject matter without deviating therefrom. Therefore, the disclosed subject matter should not be limited to any single embodiment described herein, but rather should be construed in breadth and scope in accordance with the appended claims below.

Apparatus 101, as depicted in FIG. 1A, illustrates a general overview of the outer receptacle apparatus consistent with the present disclosure. FIG. 1A shows an outer receptacle 101 having a bulb shaped head 102, a cylindrical shaft 104, and a threaded base component 106. The outer receptacle 101 may be a variety of sizes. Threaded base component 106 has grooved rings which make for easier griping and handling of the outer receptacle 101. The outer receptacle 101 may be various lengths and dimensions to accommodate various sizes of the absorbent element 205. For example, the outer receptacle 101 may have small, medium, or large sizes. The outer receptacle 101 can have dimensions ranging from a length between 5 to 20 millimeters; a width between 5 to 20 millimeters; and a height between 45 to 150 millimeters.

Regarding the outer receptacle 101, the shape may be formed in a variety of ways using various methods for delivering bodily fluids from the vaginal cavity through the inlet of the head 102, down the cylindrical shaft 104, and into the absorbent element 205 held within.

Referring now to FIG. 1B, illustrated is an example schematic of an apparatus consistent with this disclosure. FIG. 1B shows a loading cylinder 110 may have a head, a shaft, and a base. The loading cylinder 110 may be a variety of sizes. For example, the loading cylinder 110 may have small, medium, or large sizes. The loading cylinder 110 can have dimensions ranging from a length between 5 to 20 millimeters; a width between 5 to 20 millimeters; and a height between 45 to 150 millimeters to work in accordance with the outer receptacle 101.

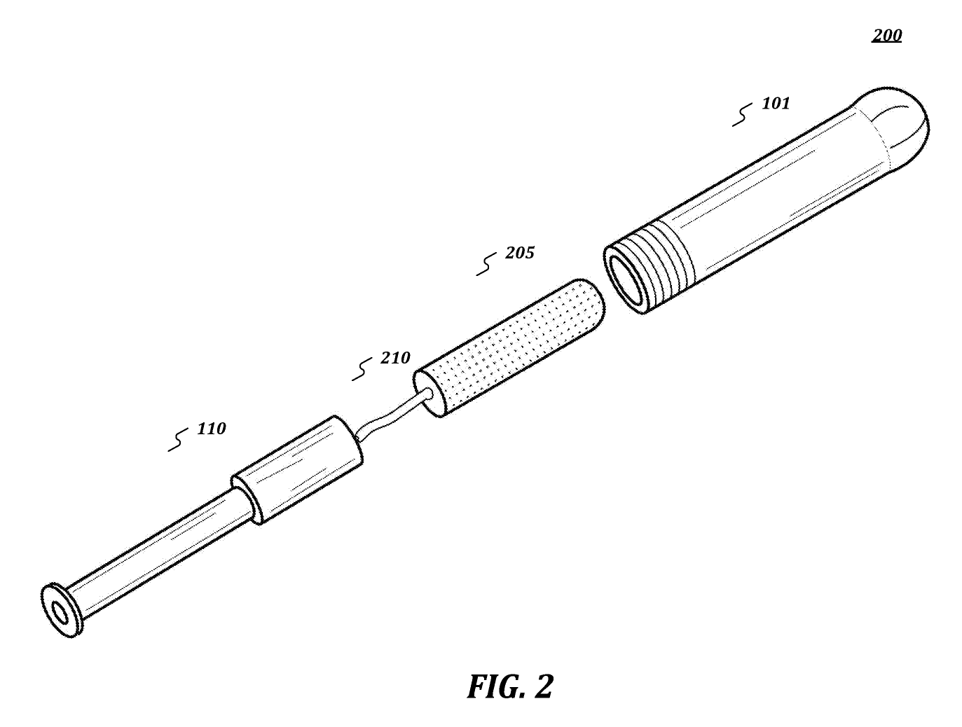

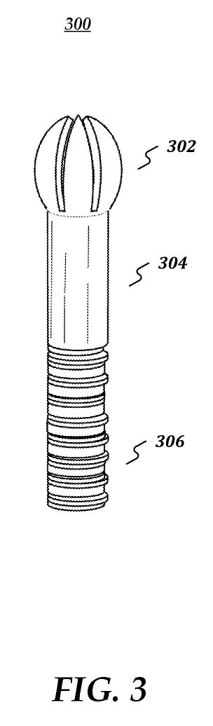

System 200, as depicted in FIG. 2, illustrates a system consistent with the present disclosure comprising the outer receptacle 101, the absorbent element 205, and the loading cylinder 110. In reference to FIG. 2, the outer receptacle 101 can be substituted with the outer receptacle 300 which is shown in FIG. 3.

The absorbent element 205, configured to absorb fluids in the vaginal cavity. Absorbent element 205 may comprise at least one of but not limited to: a round tip insert, round tip vaginal insert, tampon, low absorbency tampon, sponge, bead, gel, or compound. The absorbent element 205 can prevent leakage of fluids and/or secretions from the vaginal cavity post sexual intercourse. The present disclosure can prevent the secretions from leaking from the vaginal cavity, thus preventing stains, odors, and discomfort. Consequently, this disclosure facilitates a sanitary way to absorb secretions in the vaginal cavity post sexual activity.

The absorbent element 205 may be a variety of sizes. For example, the absorbent element 205 may have small, medium, or large sizes. The absorbent element 205 may have dimensions ranging from a length between 5 to 20 millimeters; a width between 5 to 20 millimeters; and a height between 45 to 150 millimeters to work in accordance with the outer receptacle 101.

Apparatus 301, as depicted in FIG. 3, illustrates a general overview of the outer receptacle apparatus consistent with the present disclosure. In reference to FIG. 3, at reference point 302, the apparatus 301 depicts a bulb shaped head having an opening that allows secretions to be guided towards the cylindrical shaft 304. At reference point 304, the apparatus 301 can comprise a cylindrical shaft. At reference point 306, the apparatus 301 depicts a threaded base component comprising grooved rings which make griping and handling the outer receptacle. The outer receptacle 301 can be various lengths and dimensions to accommodate various sizes of the absorbent element 205. The outer receptacle 301 can be designed having smaller dimensions in terms of length or having a greater width to accommodate a lower absorbency absorbent element 205 or a wider or narrower vaginal cavity.

This detailed description describes an absorbent element 205 of various shapes, materials, compounds, components, and aspects consistent with the present disclosure. There may be a directly proportional relationship between the size of the absorbent element 205 and the level of absorbency. For example, the greater the absorbent element 205 is in size including greater length, width, and height, the more ounces of bodily fluids can be absorbed as a higher absorbency rate. Additionally the type of material which constitutes the absorbent element 205 also determines its absorbency rate. For example, the absorbent element 205 may be a sponge, natural cotton, organic cotton, synthetic cotton, cotton, bead, gel, chemical compound, composite element, chemical composition, paste, or the like. Each element or material has its own specified or determined absorbency rate. The shape of the absorbency element also determines the efficiency of the absorbency rate. A mathematical calculation of accounting for the impact of the combination of the size, material, and shape is used to determine what we term the effective absorbency rate. The effective absorbency rate may be a whole number, decimal, scaled number, percentage, or other numerical representation.

Additionally, the components of this system, method, and apparatus may be ergonomically designed to provide maximum comfort to the user. In various embodiments, there may be plastics, ergonomic padding, soft materials and the like used to fabricate the outer receptacle 101, loading cylinder 110, absorbent element 205, and other components. Components may comprise latex, plastics, rubber, sponge, synthetic materials, nylon, cotton, organic cotton, synthetic cotton, natural cotton, fabric, cloth, wood, synthetic compounds, steel, metal rods or tubes, and other suitable materials known in the art. Components may comprise materials used to create sex toys, sex tools, and products including by not limited to smooth rubber, latex, foam, and the like.

The hollow design of the outer receptacle 101 as well as the design of the absorbent element 205 and loading cylinder 110 depicted in the figures can be ergonomically designed to give the greatest satisfaction and comfort to the user. Data from ergonomics research necessitated the design of the outer receptacle 101, loading cylinder 110, absorbent element 205, and other components in the depicted or similar shapes. As depicted later in FIG. 3, an outer receptacle 301 may be designed having smaller dimensions in terms of length or having a greater width to accommodate a lower absorbency absorbent element 205 or a wider or narrower vaginal cavity.

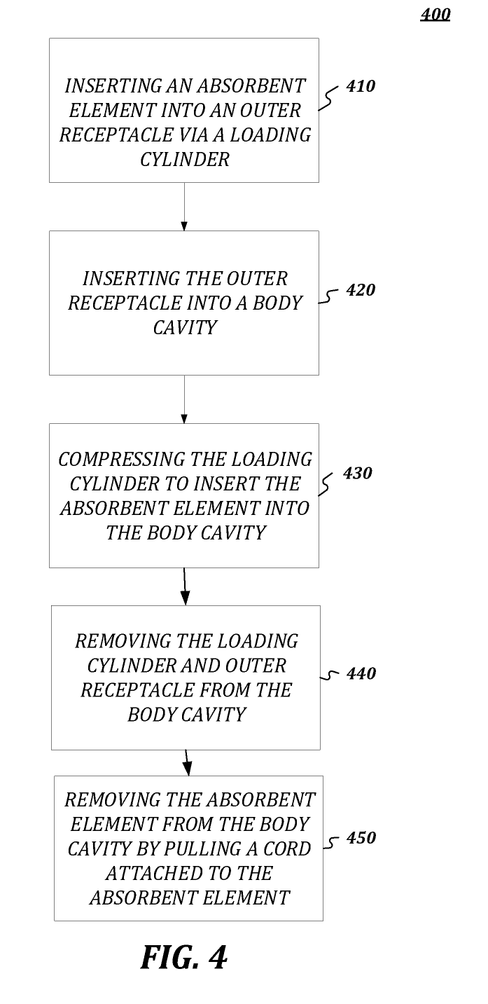

FIG. 4 illustrates a method of using the components of the system 200 depicted in FIG. 2 consistent with the present disclosure. More specifically, FIG. 4 is a flow chart setting forth the general stages involved in a method 400 consistent with an embodiment of the disclosure for providing for system 200. Method 400 can be implemented using an absorbent element 205 and cord 210 as described in more detail below with respect to FIG. 4.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein. Ways to implement the stages of method 400 will be described in greater detail below.

Methods of utilizing the absorbent element 205 may be varied from the method depicted in FIG. 4. For example, a user may desire to simply use the outer receptacle 101 and loading cylinder 110 as an applicator to directly release or place the absorbent element 205 directly into the body cavity. When used in this manner, the absorbent element 205 will be pushed through the bulb shaped head 102 of the outer receptacle 101 by the loading cylinder 110 and deposited directly into the body cavity.

In another embodiment, a user may desire to directly place the absorbent element 205 into the body cavity utilizing their hands or fingers without the assistance of the outer receptacle 101 or loading cylinder 110.

In yet another embodiment, a user may desire to use the loading cylinder 110 to deposit the absorbent element 205 into the body cavity without the assistance of the outer receptacle 101.

In yet another embodiment, a user may desire to use the outer receptacle 101 to hold the absorbent element 205. In this instance, the user may further desire to use their fingers, hand, or other device to place the absorbent element 205 into the outer receptacle 101 and body cavity without the use of the loading cylinder 110.

In an embodiment when the outer receptacle 101 remains in the body cavity while fluids are being absorbed, the outer receptacle 101 may be configured to suction out the fluids and guide them towards the absorbent element 205.

Accordingly, it should be understood that the various stages described above may be, in various embodiments, performed in arrangements that differ from the ones illustrated. Moreover, various stages may be added or removed from the flow charts without altering or deterring from the fundamental scope of the depicted methods and systems disclosed herein. A detailed description of the method described in FIG. 4 is as follows.

Method 400 may begin at starting block 410 and proceed where a user may insert an absorbent element 205 into an outer receptacle 101. For example, a user may insert an absorbent element 205 into outer receptacle 301 according to the size of the absorbent element 205 and the size of the body cavity.

From starting block 410, where the user inserted an absorbent element 205 into an outer receptacle 101, method 400 may advance to stage 420 where user may insert the outer receptacle 101 into a body cavity. For example, a user may grab the threaded base component 106 of the outer receptacle 101 using their fingers.

Once user inserts the outer receptacle 101 into the body cavity in stage 420, method 400 may continue to stage 430 where the user may compress the loading cylinder 110 to insert the absorbent element 205 into the body cavity. For example, a user may hold the outer receptacle 101 using one hand while holding the loading cylinder 110 using the other hand.

After the user compresses the loading cylinder 110 to insert the absorbent element 205 into the body cavity in stage 430, method 400 may proceed to stage 440 where the user may remove the loading cylinder 110 and outer receptacle 101 from the body cavity. For example, once the absorbent element 205 is in place within the body cavity, the user may release the loading cylinder 110 and outer receptacle 101.

After the user removes the loading cylinder 110 from the body cavity in stage 440, method 400 may proceed to stage 450 where user may remove the absorbent element 205 from the body cavity by pulling a cord 210 attached to the absorbent element 205. For example, once the absorbent element 205 has absorbed the bodily fluids during the coital intercourse, the user may remove the absorbent element 205. Once user removes the absorbent element 205 from the body cavity in stage 450, method 400 may then end at stage 450.

In another embodiment, a user may place the absorbent element 205 in the body cavity through the opening in the bulb shaped head 102 of the outer receptacle 101 using the loading cylinder 110 to thrust the absorbent element 205 into place. After absorbent element 205 is in place within the body cavity, the user may remove the loading cylinder 110 from the body cavity. After removing the loading cylinder 110 from the body cavity, the user may remove the outer receptacle 101 from the body cavity. Once the fluids are absorbed into the absorbent element 205, the user may remove the absorbent element 205 from the body cavity utilizing the cord 210 which may be a component of the absorbent element 205.

Although the stages illustrated by the flow charts are disclosed in a particular order, it should be understood that the order is disclosed for illustrative purposes only. Stages may be combined, separated, reordered, and various intermediary stages may exist. Accordingly, it should be understood that the various stages illustrated within the flow chart may be, in various embodiments, performed in arrangements that differ from the ones illustrated. Moreover, various stages may be added or removed from the flow charts without altering or deterring from the fundamental scope of the depicted methods and systems disclosed herein.

Consistent with various aspects of the present disclosure, the present disclosure may be embodied as a system, comprising: an outer receptacle, wherein the outer receptacle 101further comprises a bulb shaped head and a cylindrical shaft; an absorbent element 205, wherein the absorbent element 205 comprises a cord 210; and a loading cylinder 110.

Consistent with the present disclosure, a system, may further comprise wherein the outer receptacle 101is formed according to a predetermined length of the absorbent element 205, wherein the length is directly proportional with the absorbency rate of the absorbent element 205.

Consistent with the present disclosure, a system, may further comprise wherein the outer receptacle 101is formed according to a predetermined radius of the absorbent element 205, wherein the radius is directly proportional with the absorbency rate of the absorbent element 205.

Consistent with the present disclosure, a system, may further comprise an absorbent element 205, wherein the absorbent element 205 is at least one of a tampon, a sponge, a bead, a fabric, a compound, a gel, a paste, and a cloth cylinder.

Consistent with the present disclosure, a system, may further comprise, wherein the absorbent element 205 comprises a contraceptive. The absorbent element 205 may also be fabricated from a material that acts a contraceptive. Further, the absorbent element 205 may comprise a chemical that causes it to become a contraceptive. Yet further, the absorbent element 205 may be configured to be a contraceptive, birth control device, or the like thereof.

Consistent with the present disclosure, a system, may further comprise, wherein the absorbent element 205 comprises a medical chemical for disease prevention. The absorbent element 205 may also be fabricated from a material that acts a medical chemical for disease prevention. Further, the absorbent element 205 may comprise a chemical that causes it to release medicine(s) for disease prevention. Yet further, the absorbent element 205 may be configured to be a medical chemical for disease prevention, medical chemical for sexually transmitted infection (STI) prevention, or the like thereof.

Consistent with the present disclosure, a system, may comprise, wherein the absorbent element 205 comprises a spermicide. The absorbent element 205 may also be fabricated from a material that acts a spermicide. Further, the absorbent element 205 may comprise a chemical that causes it to act as a spermicide. Yet further, the absorbent element 205 may be configured to be a spermicide, sperm reduction agent, contraceptive, birth control device, or the like thereof.

Consistent with the present disclosure, a system, may further comprise, wherein the absorbent element 205 is configured to absorb fluids.

Consistent with the present disclosure, a system, may further comprise, wherein the absorbent element 205 absorbs fluids at an effective absorption rate.

Consistent with the present disclosure, a system, may further comprise, wherein the absorbent element 205 absorbs fluids at an absorption rate greater than a specific percentage or range from zero up to one hundred percent.

Consistent with the present disclosure, a method, may comprise inserting an absorbent element 205 into an outer receptacle 101 via a loading cylinder 110; inserting the outer receptacle 101 into a body cavity; compressing the loading cylinder 110 to insert the absorbent element 205 into the body cavity; and removing the loading cylinder 110 from the body cavity.

Consistent with the present disclosure, a method, may further comprise, removing the absorbent element 205 from the body cavity by pulling a cord 210 attached to the absorbent element 205.

Consistent with the present disclosure, a method, may further comprise wherein the body cavity is a vaginal cavity.

Consistent with the present disclosure, a method, may further comprise wherein the outer receptacle 101 further comprises at least one of a bulb shaped head, a threaded base component, and a cylindrical shaft.

Consistent with the present disclosure, a method, may further comprise wherein the absorbent element 205 is specifically configured to absorb seminal fluids.

Consistent with the present disclosure, a method, may further comprise wherein the absorbent element 205 further comprises one or more of a spermicide, a contraceptive, or a medical chemical for disease prevention.

Consistent with the present disclosure, a method, may further comprise removing at least one of the outer receptacle, loading cylinder 110, and absorbent element 205.

Consistent with the present disclosure, an apparatus, may comprise a cloth cylinder configured to absorb fluids; an absorbent element 205 configured to absorb fluids; wherein either the cloth cylinder or absorbent element 205 further comprises at least one of: a spermicide, a contraceptive, and a medical chemical for disease prevention.

Consistent with the present disclosure, an apparatus, may further comprise, wherein the apparatus absorbs fluids at an absorption rate greater than sixty percent.

Consistent with the present disclosure, an apparatus, may further comprise, wherein the apparatus is an absorbent element 205 further comprising at least one of high cotton, synthetic cotton, organic cotton, a tampon, a sponge, a bead, a fabric, and a compound.

Consistent with the present disclosure, an apparatus, may further comprise, an outer receptacle, wherein the outer receptacle 101 further comprises a bulb shaped head, a cylindrical shaft, and a threaded base component; a loading cylinder 110, wherein the loading cylinder 110 further comprises a head, a shaft, and a base; and the head of the loading cylinder 110 fitting into the threaded based component of the outer receptacle 101 in an interlocking connection that allows the head of the loading cylinder 110 to slide vertically up and down the cylindrical shaft of the outer receptacle.

Consistent with the present disclosure, an apparatus, may further comprise, wherein the loading cylinder 110 is adapted to load objects through the cylindrical shaft and the outer receptacle.

Consistent with the present disclosure, an apparatus, may further comprise, wherein the object the loading cylinder 110 is adapted to load is an absorbent element 205 further comprising at least one of a tampon, a sponge, a bead, a fabric, and a compound.

Embodiments of the present disclosure, for example, are described above with reference to block diagrams and/or operational illustrations of methods, systems, and apparatuses according to embodiments of the disclosure. The functions/acts noted in the blocks and/or components may occur out of the order as shown in any figure. For example, two blocks shown in succession may in fact be executed substantially concurrently or the blocks may sometimes be executed in the reverse order, depending upon the functionality/acts involved.

While certain embodiments of the disclosure have been described, other embodiments may exist.

While the specification includes examples, the disclosure's scope is indicated by the following claims. Furthermore, while the specification has been described in language specific to structural features and/or methodological acts, the claims are not limited to the features or acts described above. Rather, the specific features and acts described above are disclosed as example for embodiments of the disclosure.

Insofar as the description above and the accompanying drawing disclose any additional subject matter that is not within the scope of the claims below, the disclosures are not dedicated to the public and the right to file one or more applications to claims such additional disclosures is reserved.

What is claimed is:

1. A system, comprising:
an outer receptacle, wherein the outer receptacle further comprises a bulb shaped head and a cylindrical shaft;
an absorbent element, adapted to fit in the cylindrical shaft; and
a loading cylinder adapted to insert the absorbent element through the cylindrical shaft and the outer receptacle.

2. The system of claim 1, wherein the outer receptacle is formed according to a predetermined length.

3. The system of claim 1, wherein the outer receptacle comprises a predetermined radius.

4. The absorbent element of claim 1, wherein the absorbent element is one of a tampon, a sponge, a bead, a fabric, a compound, and a cloth cylinder.

5. The system of claim 1, wherein the absorbent element comprises a contraceptive.

6. The system of claim 1, wherein the absorbent element comprises a medical chemical for disease prevention.

7. The system of claim 1, wherein the absorbent element comprises a spermicide.

8. The system of claim 1, wherein the outer receptacle is formed according to at least one of a predetermined length of the absorbent element and a predetermined radius of the absorbent element wherein the length is directly proportional with the absorbency rate of the absorbent element.

9. The system of claim 1, wherein the absorbent element absorbs fluids at an effective absorption rate.

10. The system of claim 1, wherein the absorbent element absorbs fluids at an absorption rate greater than fifty percent.

11. A method, comprising:
inserting an absorbent element into a outer receptacle via a loading cylinder;
inserting the outer receptacle into a body cavity;
compressing the loading cylinder to insert the absorbent element into the body cavity;
removing the loading cylinder from the body cavity; and
removing the outer receptacle from the body cavity.

12. The method of claim 11, further comprising:
removing the absorbent element from the body cavity by pulling a cord attached to the absorbent element.

13. The method of claim 11, wherein the body cavity is a vaginal cavity.

14. The method of claim 11, wherein the outer receptacle further comprises at least one of a bulb shaped head, a threaded base component, and a cylindrical shaft.

15. The method of claim 11, wherein the absorbent element is specifically configured to absorb seminal fluids.

16. The method of claim 11, wherein the absorbent element further comprises one or more of a spermicide, a contraceptive, or a medical chemical for disease prevention.

17. The method of claim 11, further comprising removing the outer receptacle.

18. An apparatus, comprising:
an outer receptacle, wherein the outer receptacle further comprises a bulb shaped head, a cylindrical shaft, and a threaded base component;
a loading cylinder, wherein the loading cylinder further comprises a head, a shaft, and a base; and
the head of the loading cylinder fitting into the threaded based component of the outer receptacle in an interlocking connection that allows the head of the loading cylinder to slide vertically up and down the cylindrical shaft of the outer receptacle.

19. The apparatus of claim 17, wherein the loading cylinder is adapted to load objects through the cylindrical shaft and the outer receptacle.

20. The apparatus of claim 17, wherein the object the loading cylinder is adapted to load is an absorbent element further comprising at least one of a tampon, a sponge, a bead, a fabric, and a compound.
